(19) United States
(12) Patent Application Publication
(10) Pub. No.: US 2019/0371478 A1
(43) Pub. Date: Dec. 5, 2019

(54) SYSTEM AND METHOD FOR AGGREGATING PATIENT MEDICAL DATA ACQUIRED FROM DISPARATE SOURCES

(71) Applicant: Inspire Medical Systems, Inc., Golden Valley, MN (US)

(72) Inventors: John Rondoni, Plymouth, MN (US); David Todd Dieken, Minneapolis, MN (US)

(21) Appl. No.: 16/419,467

(22) Filed: May 22, 2019

Related U.S. Application Data
(60) Provisional application No. 62/678,650, filed on May 31, 2018.

(51) Int. Cl. G16H 80/00; G16H 50/50; G16H 40/67; G16H 10/60

(52) U.S. Cl. CPC G16H 80/00; G16H 10/60; G16H 40/67; G16H 50/50

(57) ABSTRACT

Methods and systems provide for interrogating a medical device associated with a patient to obtain device data using a first processor, and transmitting the device data from the first processor to a remote server. Patient-generated data is produced using a second processor and transmitted from the second processor to the remote server. Clinician-generated data is produced using a third processor and transmitted from the third processor to the remote server. The remote server aggregates the device data and one or both of the patient-generated data and the clinician-generated data to produce aggregated patient data. The aggregated patient data is accessed at the remote server by at least one of the first, second, and third processors or a separate processor. The aggregated patient data is displayed on a display coupled to at least one of the first, second, and third processors or the separate processor.

### RELATED PATENT DOCUMENTS

This application claims the benefit of Provisional Patent Application Ser. No. 62/678,650 filed on May 31, 2018, to which priority is claimed pursuant to 35 U.S.C. § 119(e), and which is incorporated herein by reference in its entirety.

### SUMMARY

Embodiments are directed to a method comprising interrogating a medical device associated with a patient to obtain device data using a first processor, and transmitting the device data from the first processor to a remote server. The method comprises producing patient-generated data using a second processor, and transmitting the patient-generated data from the second processor to the remote server. The method also comprises producing clinician-generated data using a third processor, and transmitting the clinician-generated data from the third processor to the remote server. The method further comprises aggregating, by the remote server, the device data and one or both of the patient-generated data and the clinician-generated data to produce aggregated patient data. The method comprises accessing the aggregated patient data at the remote server by at least one of the first, second, and third processors or a separate processor. The method also comprises displaying the aggregated patient data on a display coupled to at least one of the first, second, and third processors or the separate processor.

Embodiments are directed to a method comprising interrogating a therapy device associated with a patient to obtain therapy device data using a first processor, and transmitting the therapy device data from the first processor to a remote server. The method comprises producing patient-generated data using a second processor, and transmitting the patient-generated data from the second processor to the remote server. The method also comprises producing clinician-generated data using a third processor, and transmitting the clinician-generated data from the third processor to the remote server. The method further comprises aggregating, by the remote server, the therapy device data and one or both of the patient-generated data and the clinician-generated data to produce aggregated patient data. The method comprises accessing the aggregated patient data at the remote server by at least one of the first, second, and third processors or a separate processor. The method also comprises displaying the aggregated patient data on a display coupled to at least one of the first, second, and third processors or the separate processor.

Embodiments are directed to a method comprising interrogating a therapy device implantable in a patient to obtain device data using a first processor of a patient remote, and transmitting the device data from the patient remote to a remote server. The method comprises producing patient-generated data using a second processor, and transmitting the patient-generated data from the second processor to the remote server. The method also comprises producing clinician-generated data using a third processor, and transmitting the clinician-generated data from the third processor to the remote server. The method further comprises aggregating, by the remote server, the device data and one or both of the patient-generated data and the clinician-generated data to produce aggregated patient data. The method comprises accessing the aggregated patient data at the remote server by at least one of the first, second, and third processors or a separate processor. The method also comprises displaying the aggregated patient data on a display coupled to at least one of the first, second, and third processors or the separate processor.

Embodiments are directed to a system comprising a first processor configured to interrogate a medical device associated with a patient to obtain device data, a second processor configured to produce patient-generated data, and a third processor configured to produce clinician-generated data. A remote server is communicatively coupled to the first, second, and third processors. The remote server is configured to receive, in memory of the remote server, the device data, the patient-generated data, and the clinician-generated data respectively from the first, second, and third processors. The remote server is configured to aggregate the device data and one or both of the patient-generated data and the clinician-generated data to produce aggregated patient data. The remote server is also configured to grant access to the aggregated patient data by at least one of the first, second, and third processors or a separate processor. The remote server is further configured to format the aggregated patient data for presentation on a display coupled to at least one of the first, second, and third processors or the separate processor.

Embodiments are directed to a system comprising a first processor configured to interrogate a therapy device associated with a patient to obtain therapy device data, a second processor configured to produce patient-generated data, and a third processor configured to produce clinician-generated data. A remote server is communicatively coupled to the first, second, and third processors. The remote server is configured to receive, in memory of the remote server, the therapy device data, the patient-generated data, and the clinician-generated data respectively from the first, second, and third processors. The remote server is configured to aggregate the therapy device data and one or both of the patient-generated data and the clinician-generated data to produce aggregated patient data. The remote server is also configured to grant access to the aggregated patient data by at least one of the first, second, and third processors or a separate processor. The remote server is further configured to format the aggregated patient data for presentation on a display coupled to at least one of the first, second, and third processors or the separate processor.

Embodiments are directed to a system comprising a remote server. The remote server comprises a communication interface configured to communicate with a first processor configured to produce medical device data, a second processor configured to produce patient-generated data, and a third processor configured to produce clinician-generated data. The remote server comprises memory configured to store the medical device data received from the first processor, the patient-generated data received from the second processor, and the clinician-generated data received from the third processor. The remote server also comprises a data transformation module and a server processor coupled to the communication interface, the memory, and the data transformation module. The server processor is configured to aggregate the medical device data and one or both of the patient-generated data and the clinician-generated data to produce aggregated patient data. The server processor is also configured to grant access to the aggregated patient data by at least one of the first, second, and third processors or a separate processor. The server processor is further configured to cooperate with the data transformation module to display the aggregated patient data on a display coupled to at least one of the first, second, and third processors or the separate processor.

Embodiments are directed to a system comprising a remote server. The remote server comprises a communication interface configured to communicate with a first processor configured to produce therapy device data, a second processor configured to produce patient-generated data, and a third processor configured to produce clinician-generated data. The remote server comprises memory configured to store the therapy device data received from the first processor, the patient-generated data received from the second processor, and the clinician-generated data received from the third processor. The remote server also comprises a data transformation module and a server processor coupled to the communication interface, the memory, and the data transformation module. The server processor is configured to aggregate the therapy device data and one or both of the patient-generated data and the clinician-generated data to produce aggregated patient data. The server processor is also configured to grant access to the aggregated patient data by at least one of the first, second, and third processors or a separate processor. The server processor is further configured to cooperate with the data transformation module to display the aggregated patient data on a display coupled to at least one of the first, second, and third processors or the separate processor.

The above summary is not intended to describe each disclosed embodiment or every implementation of the present disclosure. The figures and the detailed description below more particularly exemplify illustrative embodiments

### BRIEF DESCRIPTION OF THE DRAWINGS

Throughout the specification reference is made to the appended drawings, where like reference numerals designate like elements, and wherein:

FIG. 1 illustrates a multiplicity of representative sources of patient-related data, each of which produces different medical data about a patient;

FIG. 2 illustrates a system for aggregating patient data acquired from disparate sources in accordance with various embodiments;

FIG. 3 illustrates a method for aggregating patient data acquired from disparate sources in accordance with various embodiments;

FIG. 4 illustrates a system for aggregating patient data acquired from disparate sources in accordance with various embodiments;

FIG. 5 shows a number of devices or systems that may be used to transmit data from a patient medical device to a remote server in accordance with various embodiments;

FIG. 6 shows a number of devices or systems that may be used to transmit data from a patient medical device to a remote server in accordance with various embodiments;

FIG. 7 shows a number of devices or systems that may be used to transmit data from a patient medical device to a remote server in accordance with various embodiments;

FIG. 8 illustrates a representative patient medical device configured to produce device data associated with a particular patient in accordance with various embodiments;

FIG. 9 illustrates a representative physiologic sensor configured to produce sensor data associated with a particular patient in accordance with various embodiments;

FIG. 10 illustrates a system for aggregating patient data acquired from disparate sources in accordance with various embodiments;

FIGS. 11-14 are representative examples of aggregated patient data for a patient acquired from disparate sources and generated by the remote server for a specific span of time in accordance with various embodiments; and

FIG. 15 shows a representative wireless programmer configured to communicate with an implantable patient therapy device in accordance with various embodiments.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

In the following description, reference is made to the accompanying set of drawings that form a part of the description hereof and in which are shown by way of illustration several specific embodiments. It is to be understood that other embodiments are contemplated and may be made without departing from the scope of the present disclosure. The following detailed description, therefore, is not to be taken in a limiting sense.

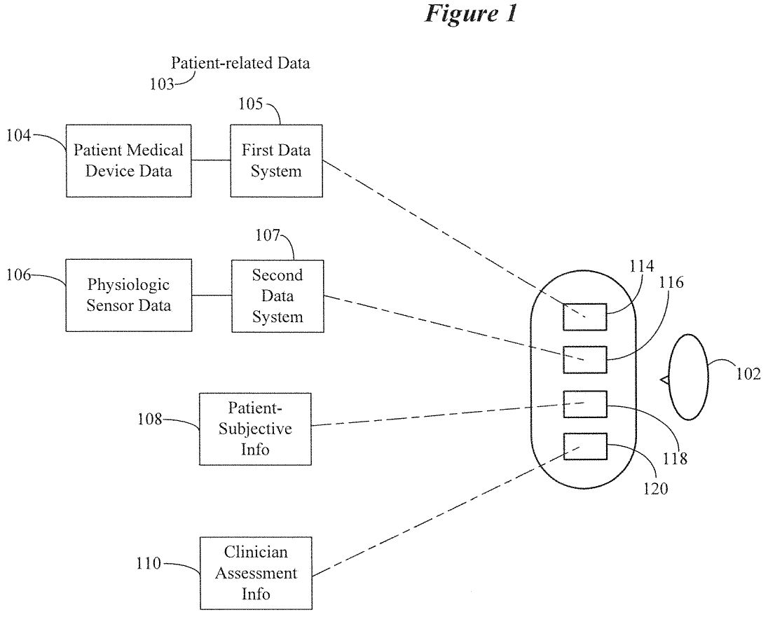

Comprehensive medical care of a patient involves the assimilation of patient-related data generated from disparate data sources by a clinician. The complete set of relevant medical data for a patient is not presently available from any one system for display to a clinician and/or a patient. For example, and with reference to FIG. 1, some patient-related data 103 relevant to providing medical care for a patient is presently acquired, stored, and outputted from disparate data systems. Other patient-related data 103 is generated by a clinician and/or a patient as a paper report, which is appended to a physical file associated with a patient.

FIG. 1 illustrates a multiplicity of representative sources of patient-related data 103, each of which produces different medical data about a patient which needs to be assimilated by a clinician 102 when providing comprehensive care for a patient. The patient-related data 103 can include patient medical device data 104 acquired from a medical device prescribed to the patient. The patient medical device data 104 is managed by a first data system 105, which can generate a medical device report 114 viewable by the clinician 102. The patient-related data 103 can also include physiologic sensor data 106 acquired from one or more physiologic sensors used to monitor one or more physiologic conditions of the patient. The physiologic sensor data 106 is managed by a second data system 107, which can generate a physiologic sensor report 116 viewable by the clinician 102. Traditionally, the first and second data systems 105, 107 are disparate systems that are communicatively isolated from one another. The patient-related data 103 can further include patient-subjective information 108 produced by the patient, typically in paper form (e.g., answers to a questionnaire and/or questions posed by a clinician). The patient-subjected information 108 can be viewed by the clinician 102 in the form of a report 118. The patient-related data 103 can also include clinician assessment information 110 produced by the clinician 102, typically in the form of a paper report 120 (e.g., notes, reports, observations).

In the illustrative traditional scenario shown in FIG. 1, the clinician 102 is required to view and evaluate separate reports 114, 116 118, 120 made available in physical form (paper) or, in some cases, as separate outputs viewable on a computer monitor. When viewable on a computer monitor, the clinician 102 typically needs to access different systems in order to view the desired patient-related data 103. In either printed or monitor-displayed form, the clinician 102 must manually evaluate each separate type of patient-related data 103, including manually assessing correlations between the disparate types of patient-related data 103. Moreover, conventional approaches to managing patient-related data 103 makes it difficult, if not impossible, to provide clinician access to relevant historical patient-related data 103 stored in disparate data systems and physical files.

Embodiments of the disclosure are directed to systems and methods that provide for the aggregation of patient medical data generated from disparate data sources for storage and access from a centralized data resource. The centralized aggregation, storage, and processing of patient medical data acquired from disparate sources for a particular patient provides for more relevant data (current and/or historical) to be made available to a clinician and/or a patient. Data that can enhance care and treatment of a patient is referred to herein as patient medical data. For example, patient medical data may exclude various types of patient-related data, such as insurance information and other patient data unrelated to the care and treatment of the patient. Patient medical data can include data acquired from one or more diagnostic and/or therapy devices (also referred to herein as medical device data or device data) and/or data acquired from one or more physiologic sensors that sense and/or monitor a physiologic condition of a patient (also referred to herein as physiologic sensor data or sensor data).

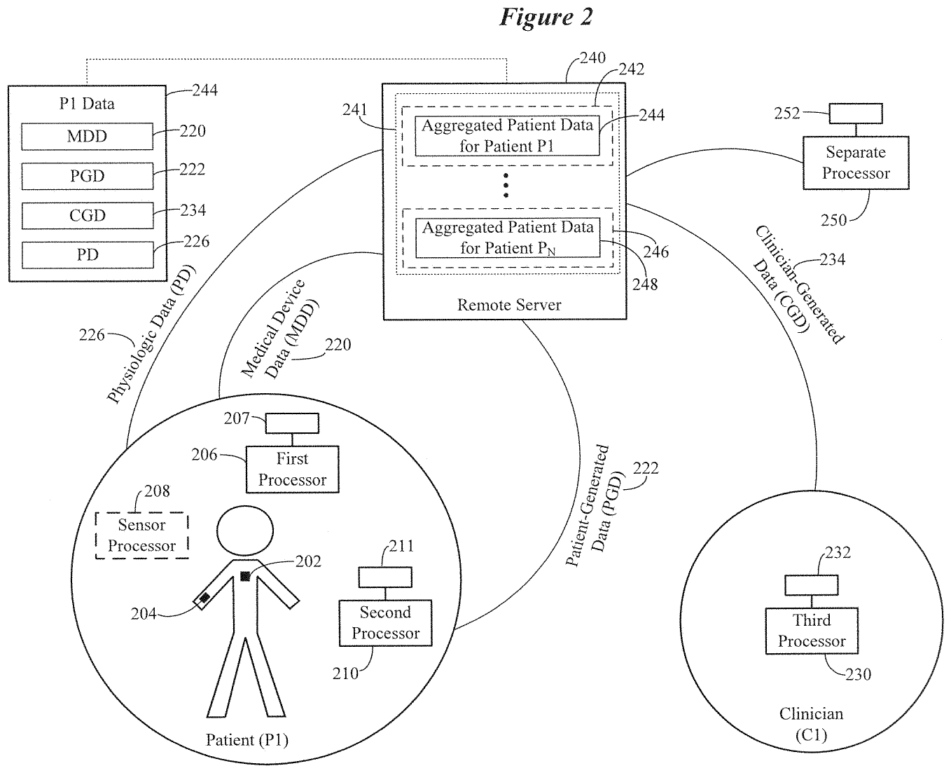

FIG. 2 illustrates a system for aggregating patient data acquired from disparate sources in accordance with various embodiments. In the representative system scenario shown in FIG. 2, a patient P1 is being cared for by a clinician C1. It is understood that, although a single clinician C1 is shown, the patient P1 may be cared for by a number of clinicians, some of whom may be affiliated or unaffiliated with respect to a medical practice. The patient P1 has been prescribed or recommended a patient medical device 202 that generates data useful to the assessment and care of the patient P1 by the clinician C1. Although shown as a single medical device 202, it is understood that the patient P1 can be prescribed or recommended multiple patient medical devices 202, each of which can generate data useful to the assessment and care of the patient P1 by the clinician C1.

In some embodiments, the patient medical device 202 can be a device subject to medical regulatory body approval (e.g., FDA approval). In other embodiments, the patient medical device 202 can be a consumer device, such as a wearable, which is not subject to medical regulatory body approval. The patient medical device 202 can be an external medical device, an implantable medical device or a combination of external and implantable medical devices. In some embodiments, the patient medical device 202 can be a diagnostic device capable of sensing and monitoring one or more physiologic signals or conditions of the patient. For example, a diagnostic device that generates patient medical data can be configured to sense and monitor one or more of oxygen saturation (e.g., via a pulse oximeter), sleep stage, respiration, snoring, posture (e.g., sleeping position, such as left, right, prone, supine), brain activity (e.g., electroencephalogram, EEG), muscle activity (e.g., electromyogram, EMG), glucose level, heart mechanical activity (e.g., heart sounds, seismocardiogram, SCG), heart electrical activity (electrocardiogram, ECG), heart rate, heart rate variability, blood pressure, temperature, and nerve activity. In other embodiments, the patient medical device 202 can be a therapy delivery device capable of delivering a therapy to the patient. For example, a therapy delivery device that generates patient medical data can be an implantable pulse generator, neurostimulator, cardiac pacemaker, resynchronizer, cardioverter/defibrillator, drug administration device (e.g., drug pump, external or implantable), diaphragm stimulator, bladder stimulator, cochlear implant, hearing aid, muscle stimulator or other type of stimulation device. As was discussed above, the patient P1 may be prescribed or recommended one or more patient medical devices 202, each of which can generate diagnostic device data and/or therapy device data.

In some embodiments, the patient medical device 202 can be a combination of two or more patient medical devices, and the medical device data generated by the combined patient medical device 202 is a combination of medical device data (MDD 220) produced by each of the patient medical devices. In other embodiments, the patient medical device 202 can be a combination of one or more patient medical devices and one or more physiologic sensors, with each of the patient medical devices and physiologic sensors producing data that is combined to define medical device data (MDD 220). In further embodiments, the patient medical device 202 can be a combination of disparate physiologic sensors each producing data that is combined to define medical device data (MDD 220). For example, a patient medical device 202 can include an array of sensors (e.g., respiration, oxygen saturation, EEG, etc.) that together define a home sleep test.

In the illustrative scenario shown in FIG. 2, the patient medical device 202 generates and stores various types of medical device data (MDD) 220. At some point in time, the patient medical device 202 is communicatively coupled to a first processor 206. In some embodiments, the first processor 206 is a processor of a medical system (e.g., at a medical clinic or in the patient's home) configured to interrogate the patient medical device 202. For example, the patient medical device 202 can be interrogated using a clinician programmer during a visit by the patient P1 to his or her clinician C1. The patient medical device 202 can also be interrogated via a medical system (e.g., bedside system) at the patient's home, such as on a daily basis. In other embodiments, the first processor 206 is a processor of a patient remote, which is configured to communicate with the patient medical device 202. In some implementations, the patient remote can be used to adjust one or more operating parameters of the patient medical device 202. In further embodiments, the first processor 206 is a processor of a consumer electronic device configured to communicate with the patient medical device 202. Communication between the patient medical device 202 and the first processor 206 can be facilitated via a wired link, a wireless link, or a combination of wired and wireless links.

The first processor 206 is also communicatively coupled to a remote server 240. The remote server 240 is typically a cloud data storage resource accessed via the Internet. In some embodiments, the remote server 240 can be a networked data storage resource accessed via private communication infrastructure. Communication between the first processor 206 and the remote server 240 can be facilitated by one or more devices (e.g., a modem, a router) and via a wired link, a wireless link, or a combination of wired and wireless links. Medical device data 220 acquired by the first processor 206 is transmitted (e.g., uploaded) to a secured location 242 in memory 241 of the remote server 240. In some embodiments, a user (e.g., clinician, patient, or patient caregiver) of the first processor 206 is authenticated prior to uploading the medical device data 220 to the remote server 240. The medical device data 220 may be encrypted such that only the remote server 240 can decrypt the medical device data 220.

The secured location 242 is allocated to store and aggregate various types of patient medical data acquired from disparate data sources for patient P1. In general, the remote server 240 is configured to store and aggregate patient medical data acquired from disparate data sources for any number of patients. For example, secured location 246 in the remote server memory 241 is allocated to store and aggregate various types of patient medical data 248 for patient P_{N}. It is understood that the remote server 240 is configured to grant access to a particular secured location associated with a particular patient only to users who are authorized (e.g., authenticated) to access aggregated patient medical data for the particular patient. For example, the remote server 240 may grant access to the aggregated patient data for patient P1 stored in secured location 242 to the patient P1 and/or one or more clinicians who are caring for the patient P1.

The patient P1 can use a second processor 210 to produce patient-generated data (PGD) 222. The second processor 210 can be a medical device (e.g., medical system at a clinic or a home medical system) or a consumer device (e.g., a PC, tablet, phablet or smartphone). The patient P1 can input information regarding the patient's subjective health, such as general wellness and specific parameters such as tiredness level. The patient P1 may also input answers to a questionnaire (e.g., Epworth Sleepiness Scale) and/or one or more open-ended questions. The second processor 210 is communicatively coupled to the remote server 240 via one or more devices (e.g., a modem, a router) and a wired link, a wireless link, or a combination of wired and wireless links. The patient-generated data 222 is transmitted (e.g., uploaded) to the secured location 242 in memory 241 of the remote server 240 via the second processor 210, assuming the patient P1 has been granted access to the secured location 242. In various embodiments, the patient P1 and/or the patient's caregiver can be authenticated prior to uploading the patient-generated data 222 to the remote server 240. The patient-generated data 222 may be encrypted, such that only the remote server 240 can decrypt the patient-generated data 222. In some embodiments, the patient-generated data 222 can be produced using the first processor 206, rather than the second processor 210. In such embodiments, the patient-generated data 222 and the medical device data 220 can be transmitted (e.g., uploaded) from the first processor 206 to the secured location 242 of the remote server 240.

The clinician C1 can use third processor 230 to produce clinician-generated data (CGD) 234. The third processor 230 can be a medical device (e.g., medical system at a clinic) or a consumer device (e.g., a PC, laptop, tablet, phablet or smartphone). The clinician-generated data 234 can include outcome data, annotations, and/or notes generated by the clinician relating to the care of the patient P1. The clinician-generated data 230 can also include responses to a single question or answers to a multi-part questionnaire. The third processor 230 is communicatively coupled to the remote server 240 via one or more devices (e.g., a modem, a router) and a wired link, a wireless link, or a combination of wired and wireless links. The clinician-generated data 234 is transmitted (e.g., uploaded) to the secured location 242 in memory 241 of the remote server 240 via the third processor 230, assuming the clinician C1 has been granted access to the secured location 242. In various embodiments, the clinician C1 can be authenticated prior to uploading the clinician-generated data 234 to the remote server 240. The clinician-generated data 234 may be encrypted, such that only the remote server 240 can decrypt the clinician-generated data 234.

Physiologic data (PD) 226 can be generated by one or more physiologic sensors coupled to or situated in proximity to the patient P1. As shown in FIG. 2, a physiologic sensor 204 is coupled to the patient P1 and configured to sense and/or monitor one or more physiologic conditions of the patient P1. The physiologic data 226 can include data relevant to the treatment of a medical condition, such as AHI (Apnea Hypopnea Index), oxygen saturation, sleep stage, respiration, heart rate, heart rate variability, temperature, blood pressure (or data produced by a diagnostic device discussed above), and weight (via a scale). The physiologic sensor 204 can be communicatively coupled to a sensor processor 208, such as via a wired link, wireless link, or combination of wired and wireless links (e.g., USB, FireWire®, Lightning®, ISO/IEEE 11073, IEEE 802.11, Bluetooth® and/or ZigBee® link). The sensor processor 208 can be a medical device (e.g., medical system at a clinic or a home medical system) or a consumer device (e.g., a PC, tablet, phablet or smartphone). The sensor processor 208 is communicatively coupled to the remote server 240 via one or more devices (e.g., a modem, a router) and a wired link, wireless link, or combination of wired and wireless links. Physiologic data 226 acquired by the physiologic sensor 204 is transmitted (e.g., uploaded) to the secured location 242 in memory 241 of the remote server 240. In various embodiments, a user of the sensor processor 208 (e.g., patient P1 or P1's caregiver) can be authenticated prior to uploading the physiologic data 226 to the remote server 240. The physiologic data 226 may be encrypted, such that only the remote server 240 can decrypt the physiologic data 226. In some embodiments, the physiologic data 226 can be transmitted from the physiologic sensor 204 to the first processor 206 or the second processor 210 for transmission to the secured location 242 of the remote server 240.

In general, the aggregated patient data stored in the remote server 240 can be accessed by various entities that have an association with the patient P1 (e.g., the patient P1, the patient's caregiver(s), the patient's clinician(s), a medical practice caring for patient P1). The remote server 240 can implement an authentication process by which credentials of an entity requesting access to aggregated patient data stored in memory 241 are authenticated. In the illustrative scenario shown in FIG. 2, the remote server 240 may grant access by one or more of the first processor 206, second processor 210, third processor 230, and a separate processor 250 to the aggregated patient data for patient P1 stored in the secured location 242 in the remote server 244. The aggregated patient data for patient P1 stored in the secured location 242 can include current (e.g., most recently uploaded data) and/or historical medical device data 220, patient-generated data 222, clinician-generated data 234, and physiologic data 226 (or any subset of these data). Aggregated patient data stored in the secured location 242 can be transmitted from the remote server 244 to one or more of the first processor 206, second processor 210, third processor 230, and the separate processor 250. Aggregated patient data may be displayed on a display 207, 211, 232, 252 respectively coupled to the first processor 206, second processor 210, third processor 230, and the separate processor 250.

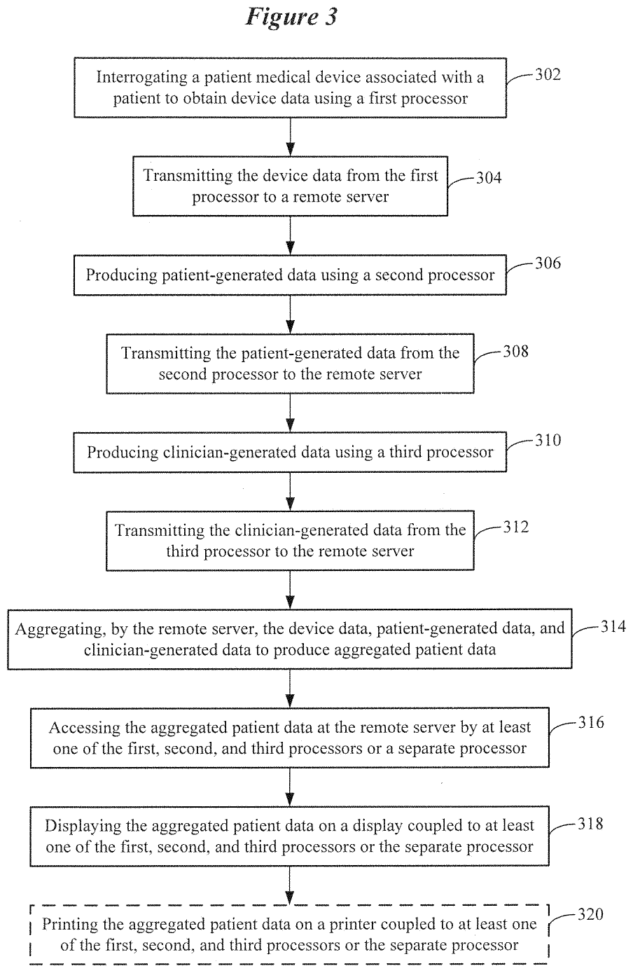

FIG. 3 illustrates a method for aggregating patient data acquired from disparate sources in accordance with various embodiments. The method illustrated in FIG. 3 involves interrogating 302 a patient medical device associated with a patient to obtain device data using a first processor, and transmitting 302 the device data from the first processor to a remote server. The method involves producing 306 patient-generated data using a second processor, and transmitting 308 the patient-generated data from the second processor to the remote server. The method also involves producing 310 clinician-generated data using a third processor, and transmitting 312 the clinician-generated data from the third processor to the remote server. In some embodiments, the physiologic data can be generated using a fourth processor, and the physiologic data can be transmitted from the fourth processor to the remote server. The method involves aggregating 314, by the remote server, the device data and one or both of patient-generated data and clinician-generated data to produce aggregated patient data. In some embodiments, the aggregated patient data can include physiologic data. The method also involves accessing 316 the aggregated patient data at the remote server by at least one of the first, second, and third processors or a separate processor. The method further involves displaying 318 the aggregated patient data on a display coupled to at least one of the first, second, and third processors or the separate processor. In some embodiments, the method can involve printing 320 the aggregated patient data on a printer coupled to at least one of the first, second, and third processors or the separate processor. It is understood that the method shown in FIG. 3 can be implemented for each of a multiplicity of patients.

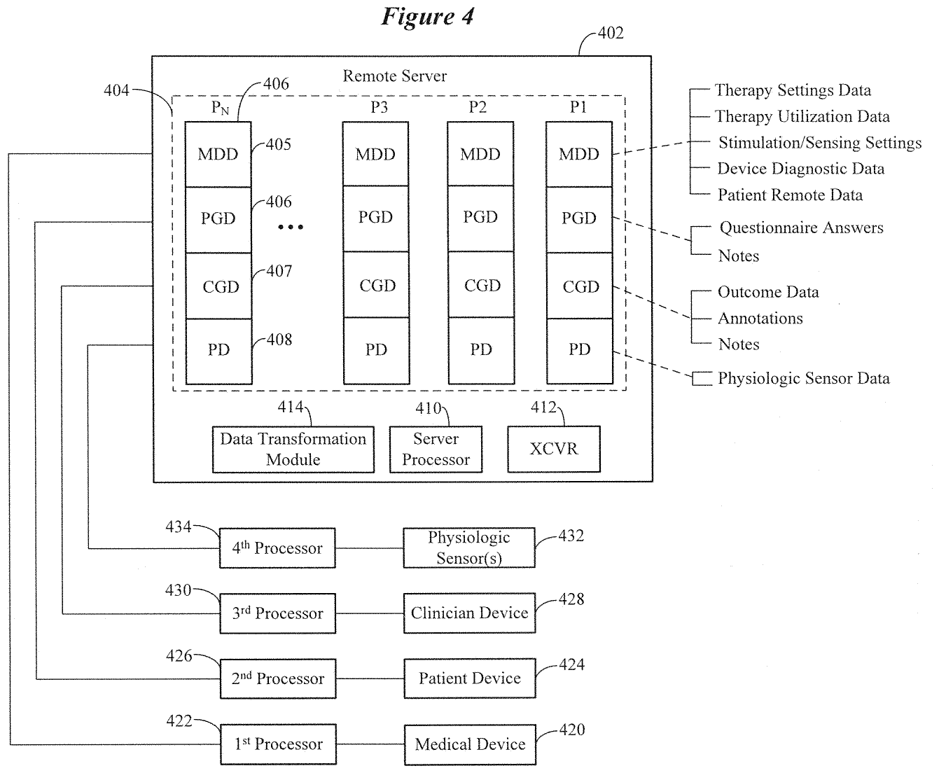

FIG. 4 illustrates a system for aggregating patient data acquired from disparate sources in accordance with various embodiments. The system illustrated in FIG. 4 includes a remote server 402 having a memory 404 configured to include a secured location 406 for storing aggregated patient data for each of a number of different patients P1-P_{N}. The remote server 402 includes a server processor 410 configured to host, deliver, and manage the resources and services provided by the remote server 402 to various client processors. The remote server 402 includes a transceiver 412 configured to facilitate communication with each of a number of client processors over a network (e.g., a LAN, WLAN) and/or an Internet connection. The remote server 402 also includes a data transformation module 414. The data transformation module 414 is configured to convert data stored in the memory 404 of the remote server 402 from one format or structure into another format or structure appropriate for a target device, such as a client processor having a display or printer resource.

In some embodiments, the server processor 402 is configured to perform storage and/or retrieval processing on one or more of the patient medical data, clinician-generated information, patient-generated information, and physiologic sensor data. For example, the server processor 402 may perform various functions, such as data format transformation, and implementation of de-duplication logic to ensure that the data sent by the same source or different sources is not duplicated. The server processor 402 can be configured to perform pre-determined processing or, in some embodiments, processing that changes over time according to various optimization means such as machine learning based on a patient's data in machine learning based on anonymized data for all patient data stored in the remote server 402. It is noted that the data storage, storage processing, and retrieval processing performed by the remote server 402 can be implemented on a medical device and/or a consumer device in addition to the remote server 402. This processing may be a subset of the processing that occurs in the remote server 402. This allows access and display of patient medical data without communication with the remote server 402 for performance or if connectivity is not present.

In the representative example shown in FIG. 4, the remote server 402 communicates with and receives patient medical data from a first processor 422, a second processor 426, a third processor 430, and a fourth processor 434 via the transceiver 412. In particular, the first processor 422 acquires medical device data (MDD) from one or more medical devices 420 associated with a particular patient (e.g., P1). The second processor 426 acquires patient-generated data (PGD) produced by a patient device 424, such as a PC, tablet, phablet or smartphone operated by the particular patient (e.g., P1). The third processor 430 acquires clinician-generated data (CGD) produced by a clinician device 428, such as a medical device or system, PC, tablet, phablet or smartphone operated by a clinician associated with the particular patient (e.g., P1). The fourth processor 434 acquires physiologic data (PD) from one or more physiologic sensors 432 coupled to or situated proximate the particular patient (e.g., P1). The patient medical data acquired from the disparate sources shown in FIG. 4 for a multiplicity of patients is stored in a secured location 406 in memory 404 allocated for each patient P1-P_{N}.

It is assumed, for purposes of illustration and not of limitation, that a patient medical device associated with patient P1 is a therapy device, such as a neurostimulation device configured to deliver a neurostimulation therapy for treating an obstructive disordered breathing condition of the patient P1. The medical device data (MDD) 405 stored in the secured location 406 for patient P1 includes one or more of therapy settings data, therapy utilization data, stimulation/sensing settings, device diagnostic data, and patient remote data. For example, therapy settings data can include programmable parameter values unique to the patient. The therapy utilization data can include the date/time therapy was turned on, off, and paused, and patient configuration such as therapy amplitude. The stimulation/sensing settings can include stimulation amplitudes, stimulation thresholds, and exhalation/inhalation sensing settings. Device diagnostic data can include data used to determine if the patient medical device operation and/or performance is as expected or designed. Device diagnostic data can include, for example, battery status, electrode status, communication device status, processor status, memory status, firmware/software version/status, pulse generator status, and fault codes.

In some embodiments, the patient remote can be configured to facilitate patient adjustment of one or more therapy settings of the therapy device 420 directly, without the need of a programmer. The patient remote includes a communication interface (wired or wireless) configured to facilitate communication with the therapy device 420. The patient remote also includes memory that can store data produced by the therapy device 420, such as therapy settings data including patient-initiated programming changes, therapy utilization data, physiologic data, and device diagnostic data. As such, the first processor 422 that interrogates the patient medical device 420 can be a processor of the patient remote. The processor of the patient remote can produce the medical device data 405 rather than, or in addition to, a separate processor (e.g., of a programmer) that interrogates the therapy device 420. In some embodiments, the patient remote can be implemented to provide the functionality of the patient remote disclosed in commonly-owned U.S. Pat. No. 9,839,786 (Rondoni et al.) and U.S. Pat. Pub. No. 2016/0193468 (Rondoni et al.), each of which is incorporated herein by reference.

The patient-generated data (PGD) 406 stored in the secured location 406 for patient P1 includes answers to a questionnaire (e.g., Epworth Sleepiness Scale) and/or one or more open-ended questions, and patient notes. The clinician-generated data (CGD) 407 stored in the secured location 4064 patient P1 includes outcome data, annotations, notes, and observations made by a clinician in connection with care given to the patient P1. The physiologic data (PD) 408 includes data relevant to the treatment of a medical condition, such as AHI (Apnea Hypopnea Index), oxygen saturation, sleep stage, respiration, heart rate, heart rate variability, temperature, and blood pressure (or data produced by a diagnostic device discussed above).

The aggregated data 405, 406, 407, 408 stored in the secured location 406 for the patient P1 can be accessed by entities associated with the patient P1, typically after successful completion of an authentication process. The aggregated data 405, 406, 407, 408 is processed by the data transformation module 414 to transform the aggregated data into a format or structure that is appropriate for a target device (e.g., a processor, a display, a printer).

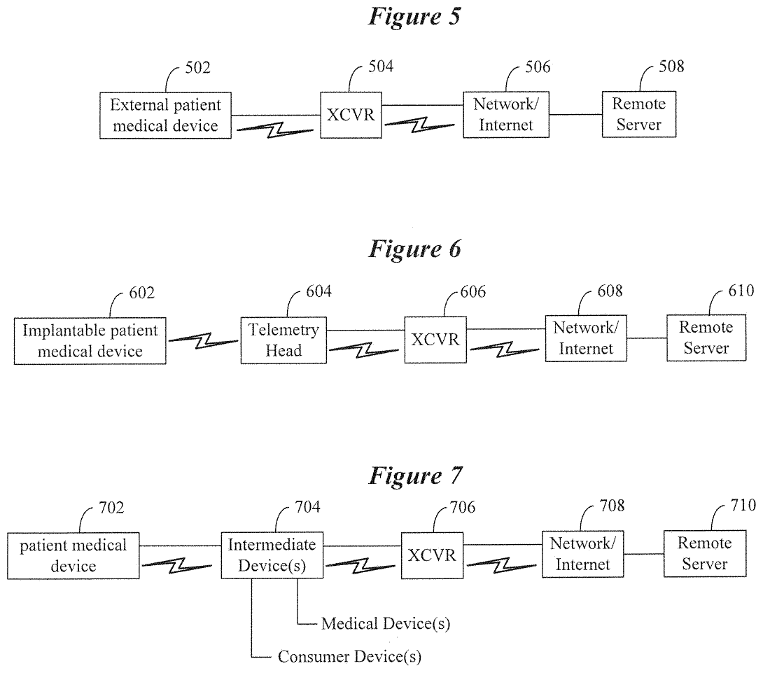

According to various embodiments, more than one device or system may be used to transmit data from a patient medical device to the remote server. For example, and with reference to FIG. 5, an external patient medical device 502 can communicate device data to a transceiver 504 via a wired or wireless link. The external patient medical device 502 can be implemented as a patient remote control, bedside communication device, bedside sensing device, wearable communication device, or wearable sensing device (e.g., pulse oximeter, blood pressure sensor, temperature sensor). The wired link can be a serial, parallel, USB, FireWire®, or Lightning® link. The wireless link can be a Bluetooth® link, a ZigBee® link, WiFi®, IEEE 802.11 or ISO/IEEE 11073 compliant communication link. The transceiver 504 communicates the device data to a network and/or the Internet 506 via a wired and/or wireless communication link (e.g., GSM, CDMA, GPRS, HDSPA). The remote server 508 receives the device data from the network or the Internet 506. The devices/systems shown in FIG. 5 may be configured to forward the device data, or may store/forward the device data as connectivity permits.

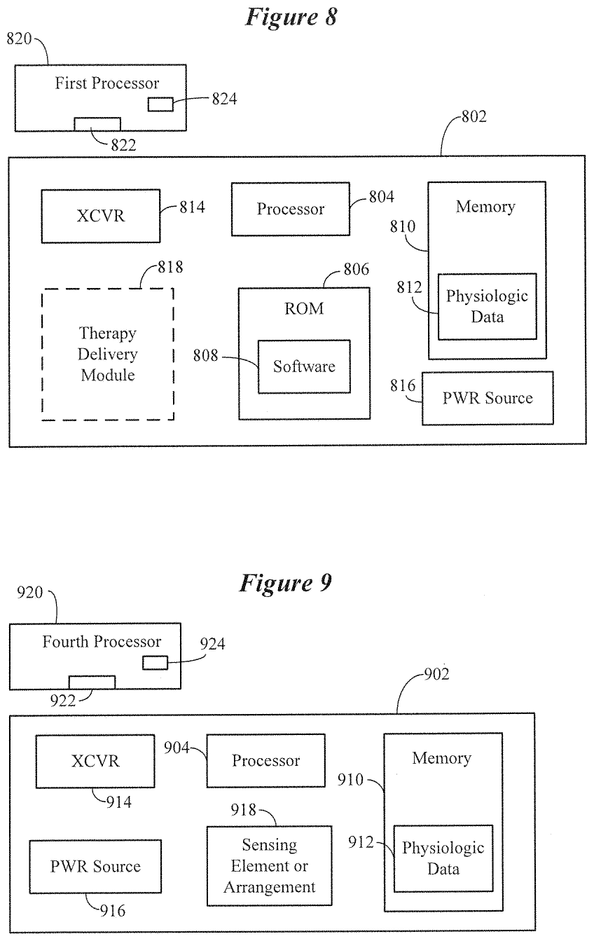

Referring to FIG. 6, an implantable patient medical device 602 can communicate device data to a telemetry head 604, which may be a near-field link (e.g., an inductive communication link or a far-field radiofrequency link such as a BLE link). The implantable patient medical device 602 can be implemented as any implantable device, including those discussed herein. The device data is communicated from the telemetry head 604 to a transceiver 606, network/Internet 608, and remote server 610 in a manner discussed above. The devices/systems shown in FIG. 6 may be configured to forward the device data, or may store/forward the device data as connectivity permits.

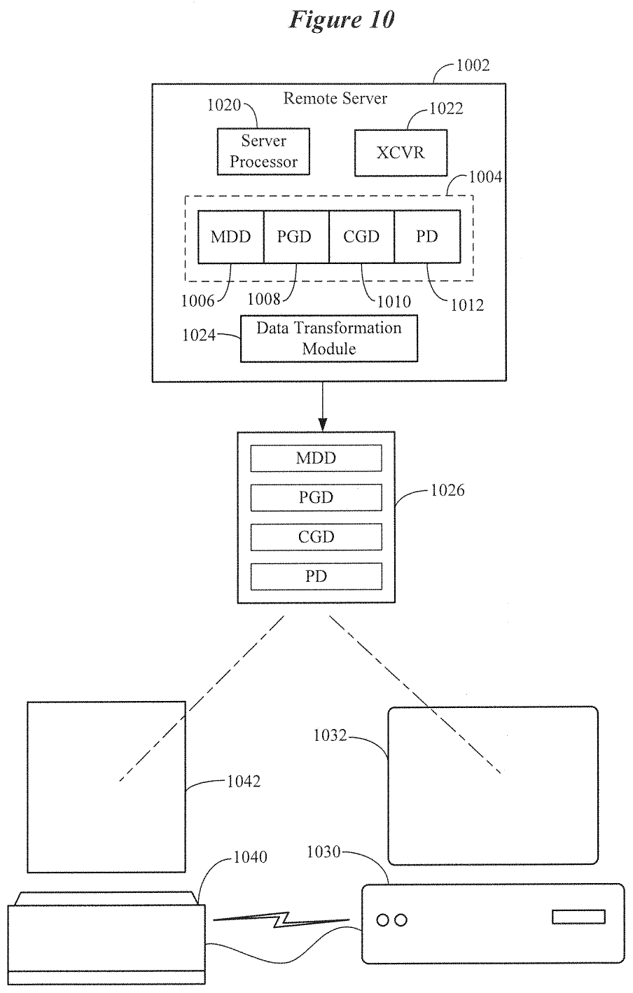

With reference to FIG. 7, a patient medical device 702 communicates device data to one or more intermediate devices 704 via a wired or wireless link. The patient medical device 702 can be an external or implantable device. The one or more intermediate devices 704 can include a medical device (e.g., patient remote, programmer), a consumer device (PC, laptop, tablet, phablet, smartphone), or a combination of medical and consumer devices. The device data acquired by the one or more intermediate devices 704 is communicated to a transceiver 706, network/Internet 708, and remote server 710 in a manner discussed above. The devices/systems shown in FIG. 7 may be configured to forward the device data, or may store/forward the device data as connectivity permits.

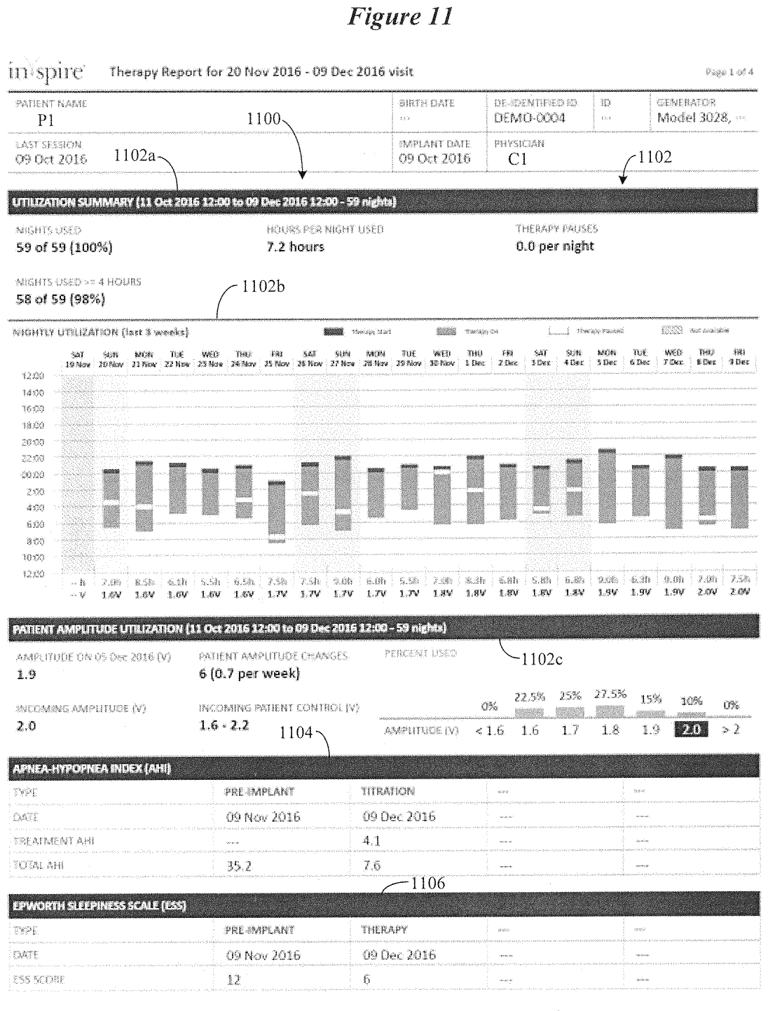

FIG. 8 illustrates a representative patient medical device configured to produce device data associated with a particular patient in accordance with various embodiments. The patient medical device 802 includes a processor 804 coupled to memory 806 and 810. The processor 804 can be a microprocessor, an embedded microprocessor, an embedded controller, or a digital signal processor (DSP), for example. The processor 804 is configured to execute program code stored as software 808 in a read-only memory (ROM) 806. The program code, when executed by the processor 804, causes the processor 804 to implement the various medical device functions described herein. The processor 804 cooperates with memory 810 (e.g., flash, SRAM) to store physiologic data 812 acquired by one or more sensors of the patient medical device 802. A power source 816, which can be rechargeable, provides power to the various components of the patient medical device 802. In some embodiments, the patient medical device 802 is a diagnostic device. In other embodiments, the patient medical device 802 is a therapy device, and includes a therapy delivery module 818. The therapy delivery module 818 can include a pulse generator coupled to an electrode arrangement, for example.

The patient medical device 802 includes a transceiver 814 which can be configured to facilitate wired or wireless communication with another device or system via any of the protocols described herein. In some embodiments, the transceiver 814 is configured to facilitate communication between the patient medical device 802 and a network or the Internet via a router and/or modem. In other embodiments, the transceiver 814 is configured to facilitate communication between the patient medical device 802 and a first processor 820 (see, e.g., FIG. 4) which, in turn, is communicatively coupled to a network and/or the Internet as previously described. The first processor 820 includes a transceiver 822 configured to communicatively coupled to the transceiver 814 of the patient medical device 802. The transceivers 814, 822 can be configured to facilitate wired or wireless communication via any of the protocols described herein. Device data generated by or acquired by the patient medical device 802 can be transferred to a memory 824 coupled to the first processor 820 via the transceivers 814, 822.

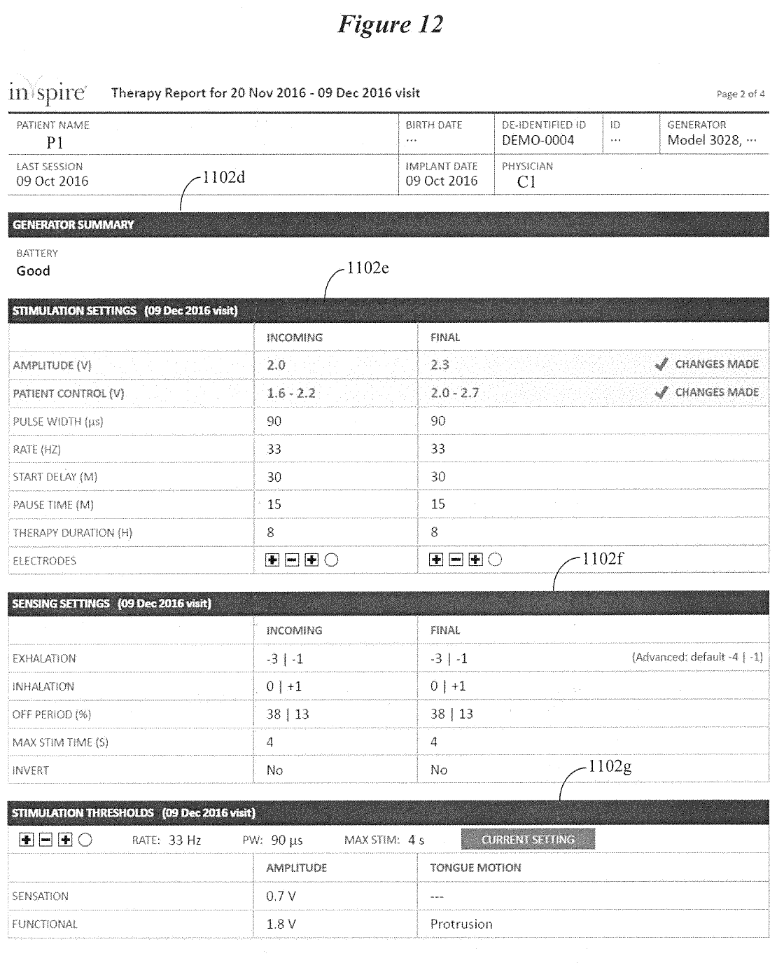

FIG. 9 illustrates a representative physiologic sensor configured to produce sensor data associated with a particular patient in accordance with various embodiments. The physiologic sensor 902 includes a processor 904 coupled to a memory 910. The processor 904 can be a microprocessor, an embedded microprocessor, an embedded controller, a digital signal processor, an ASIC, a field programmable gate array, or a programmable logic device, for example. In some embodiments, the processor 904 is configured to execute program code stored as software in a nonvolatile memory 905 (e.g., ROM) to implement the various physiologic sensor functions described herein. The processor 904 cooperates with memory 910 (e.g., flash, SRAM) to store physiologic data 912 acquired by a sensing element or arrangement 918. A power source 916, which can be rechargeable, provides power to the various components of the physiologic sensor 902.

The physiologic sensor 902 includes a transceiver 914 which can be configured to facilitate wired or wireless communication with another device or system via any of the protocols described herein. In some embodiments, the transceiver 914 is configured to facilitate communication between the physiologic sensor 902 and a network or the Internet via a router and/or modem. In other embodiments, the transceiver 914 is configured to facilitate communication between the physiologic sensor 902 and a fourth processor 920 (see, e.g., FIG. 4) which, in turn, is communicatively coupled to a network and/or the Internet as previously described. The fourth processor 920 includes a transceiver 922 configured to communicatively coupled to the transceiver 914 of the physiologic sensor 902. The transceivers 914, 922 can be configured to facilitate wired or wireless communication via any of the protocols described herein. Sensor data generated by or acquired by the physiologic sensor 902 can be transferred to a memory 924 coupled to the fourth processor 920 via the transceivers 914, 922.

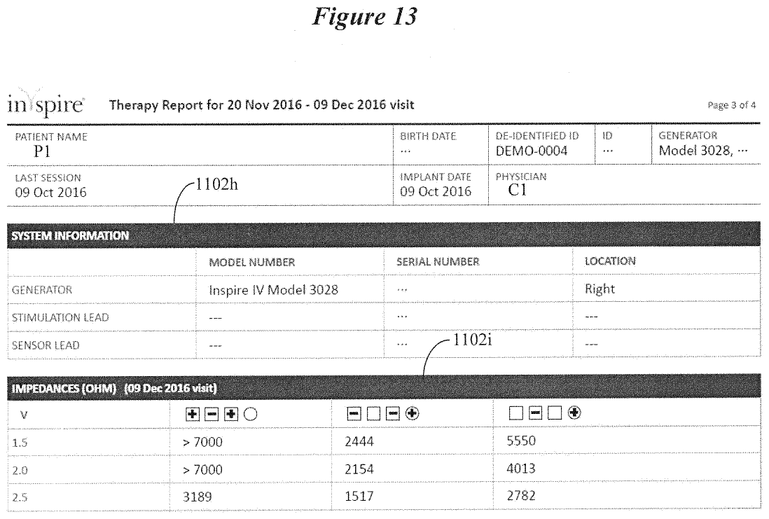

FIG. 10 illustrates a system for aggregating patient data acquired from disparate sources in accordance with various embodiments. In the embodiment shown in FIG. 10, a remote server 1002 includes a memory 1004, a server processor 1020, a transceiver 1022, and a data transformation module 1024. The remote server 1002 is configured to receive patient medical data from a multiplicity of disparate sources for each of a plurality of patients in a manner described previously. The memory 1004 is configured to store disparate patient medical data for each patient in a separate secured location as aggregated patient data. For clarity of explanation, FIG. 10 shows aggregated patient data associated with a single patient (e.g., patient P1). The aggregated patient data stored in memory 1004 includes medical device data 1006, patient-generated data 1008, clinician-generated data 1010, and physiologic data 1012.

In this illustrative example, a user (e.g., a patient or a clinician) accesses the remote server 1002 via the Internet using a modem and a processor of a personal computer 1030. It is understood that the personal computer 1030 can instead be representative of a medical system, laptop, notebook, tablet, phablet or smartphone. The personal computer 1030 is shown coupled to a display 1032 and a printer 1040. Typically, access to the remote server 1002 and to aggregated patient data for a particular patient (e.g., patient P1) is granted to the user after successfully completing an authentication process. The user may select the most relevant aggregated patient data for a particular patient and for specific point in time. The user may select the most relevant aggregated patient data for a particular patient and for a particular span of time. The most relevant patient medical data may be a subset of the aggregated patient data stored in the remote server 1002 for a particular patient. In some implementations, the server processor 1020 is configured to determine the most relevant aggregated patient data for a particular patient. The most relevant patient medical data associated with a particular patient can be data most likely to be useful for the clinician for the purpose of furthering the patient's care. For example, the most relevant patient medical data associated with a particular patient can be the newest data of each type as of the current time, the newest data for each type as of a clinician selected time, a subset of data types having data newer than a given period, a subset of data types having characteristics different from the norm, a subset of data types most often selected by the clinician, a subset of data types most recently selected by the clinician, a subset of data types most often selected by all clinicians, or a combination of these.

The user may request aggregated patient data for a specific point in time. In response, the server processor 1020 selects aggregated patient data for the particular patient from the memory 1004 for the specific date or date and time requested by the user. The data transformation module 1024 formats the aggregated patient data for the specific point in time to produce an output 1026 having a format appropriate for presentation on the display 1302 and/or to produce a report 1042 by the printer of 1040.

The user may request aggregated patient data for a specific span of time. In response, the server processor 1020 selects aggregated patient data for the particular patient from the memory 1004 for the specific span of time requested by the user. In some embodiments, the server processor 1020 is configured to automatically compute the specific span of time as an interval between patient visits to a clinician or medical practice. The span of time between patient visits is particularly relevant to the determination of whether therapy is effective and/or a particular patient is complying with therapy directives issued by a clinician. The data transformation module 1024 formats the aggregated patient data for the specific span of time to produce an output 1026 having a format appropriate for presentation on the display 1032 and/or to produce a report 1042 by the printer of 1040.

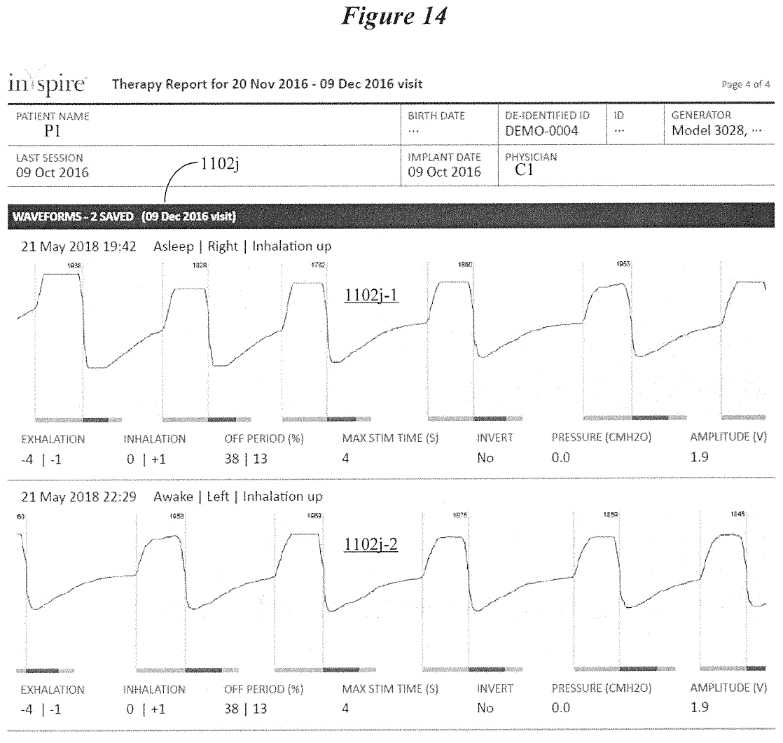

FIGS. 11-14 are representative examples of aggregated patient data transmitted from a remote server and outputted to a display or a printer coupled to a user's computing or communication device in accordance with various embodiments. The user may be the patient P1, one or more clinicians (e.g., clinician C1) who care for the patient P1, or a clinician or other personnel at a medical practice that provides care for the patient P1. The user's computing or communication device can be any of the devices and systems discussed hereinabove. In this illustrative example, the aggregated patient data for a particular patient (P1) includes medical device data acquired by the remote server from the patient's implanted neurostimulation device. The neurostimulation device in this example is configured to deliver a neurostimulation therapy for treating an obstructive disordered breathing condition of the patient P1. The medical device data stored in the implanted neurostimulation device and transmitted to the remote server can be accessed by a clinician programmer and/or a patient remote via a near-field link or a far-field link. In some embodiments, the medical device data stored in the implanted neurostimulation device and transmitted to the remote server can be accessed by a consumer electronic device (e.g., smartphone) running a medical device app and equipped with an appropriate transceiver configured to communicate with the implanted neurostimulation device (e.g., via a far-field link, such as a BLE link).

FIG. 11 is a representative example of aggregated patient data 1100 for patient P1 acquired from disparate sources and generated by the remote server for a specific span of time. The aggregated patient data 1100 includes medical device data 1102 and one or both of clinician-generated data 1104 and patient-generated data 1106. The medical device data 1102 shown in FIG. 11 includes therapy utilization summary data 1102a, nightly therapy utilization data 1102b, and patient remote data 1102c. The therapy utilization summary data 1102a provides a utilization summary for the specific span of time, including number and percentage of nights therapy was used by the patient P1, hours per night therapy was used by the patient P1, therapy pauses initiated by the patient P1, and the number and percentage of nights therapy was used by the patient P1 in excess of 4 hours. The nightly therapy utilization data 1102b provides nightly therapy utilization by the patient P1 over the last 3 weeks of the specific span of time. The nightly therapy utilization data 1102b is presented as a stacked bar graph showing therapy start time and start delay duration, the duration therapy was on, and the duration therapy was paused by the patient P1. It is noted that the therapy start time, the duration therapy is on, and the duration therapy is paused is controlled by the patient P1 using the patient remote. Under each nightly bar graph, the total duration of therapy and the stimulation amplitude are presented in numerical form.

The patient remote data 1102c includes patient amplitude utilization data over the specific span of time. The patient amplitude utilization data is based on the patient's interaction with the patient remote, which provides the patient the ability to adjust the stimulation amplitude within an amplitude range established by the clinician C1. The patient remote data 1102c includes the incoming amplitude at the time of a clinician visit and the previous amplitude set by the patient P1. The number of patient amplitude changes during the specific span of time is also included, as is the average number of patient amplitude changes per week. The incoming patient amplitude control range established by the clinician C1 is also provided. Also included is a histogram of utilization (percentage) for each of the amplitude increments falling within the patient amplitude control range established by the clinician C1.

The clinician-generated data 1104 includes Apnea-Hypopnea Index (AHI) data generated by the clinician C1 during a sleep study of the patient P1 at a sleep laboratory. In some cases, the clinician-generated data 1104 is manually generated data. In other cases, the clinician-generated data 1104 is computer-generated data or a combination of computer and manually generated data. For example, AHI can be generated manually or via a computer or the implanted neurostimulation device. The clinician-generated data 1104 includes pre-implant data and titrated data. The pre-implant data includes the patient's AHI prior to implant with no therapy being delivered. The titrated data includes the patient's AHI after titration of the neurostimulation therapy by the clinician C1 (or a clinician at the sleep laboratory). The clinician-generated data 1104 provides outcome data when therapy is active and when therapy is inactive for purposes of comparison. The clinician-generated data 1104 can also provide comparative outcome data associated with one or more different therapy settings, such as outcome data at different therapy amplitudes.

The patient-generated data 1106 includes subjective health data produced by the patient P1. The patient-generated data 1106 includes the patient's score on a health questionnaire, in this case an Epworth Sleepiness Scale (ESS) questionnaire. The patient-generated data 1106 includes the patient's pre-implant ESS score and ESS score with therapy delivered to the patient P1.

Depending on the user's needs, additional medical device data 1102 can be retrieved from the remote server for output to a display or a printer coupled to the user's computing or communication device. FIGS. 12-14 are representative examples of additional medical device data 1102 that can be retrieved from the remote server. The medical device data 1102 shown in FIGS. 12 and 13 includes system information about the pulse generator of the implanted neurostimulation device. The system information includes generator summary data 1102d (e.g., battery status), generator/stimulation lead/sensor lead information 1102h, and impedance data 1102i for various electrode configurations. FIG. 12 also shows incoming and final (clinician modified) stimulation settings 1102e, sensing settings 1102f, and stimulation thresholds 1102g. Referring to the stimulation settings 1102e, changes made to the amplitude and patient control settings are highlighted (accentuated with a check mark). FIG. 14 shows respiration waveforms 1102j generated by data acquired from the implanted neurostimulation device when the patient P1 was asleep (waveform 1102j-1) and when the patient P1 was awake (waveform 1102j-2).

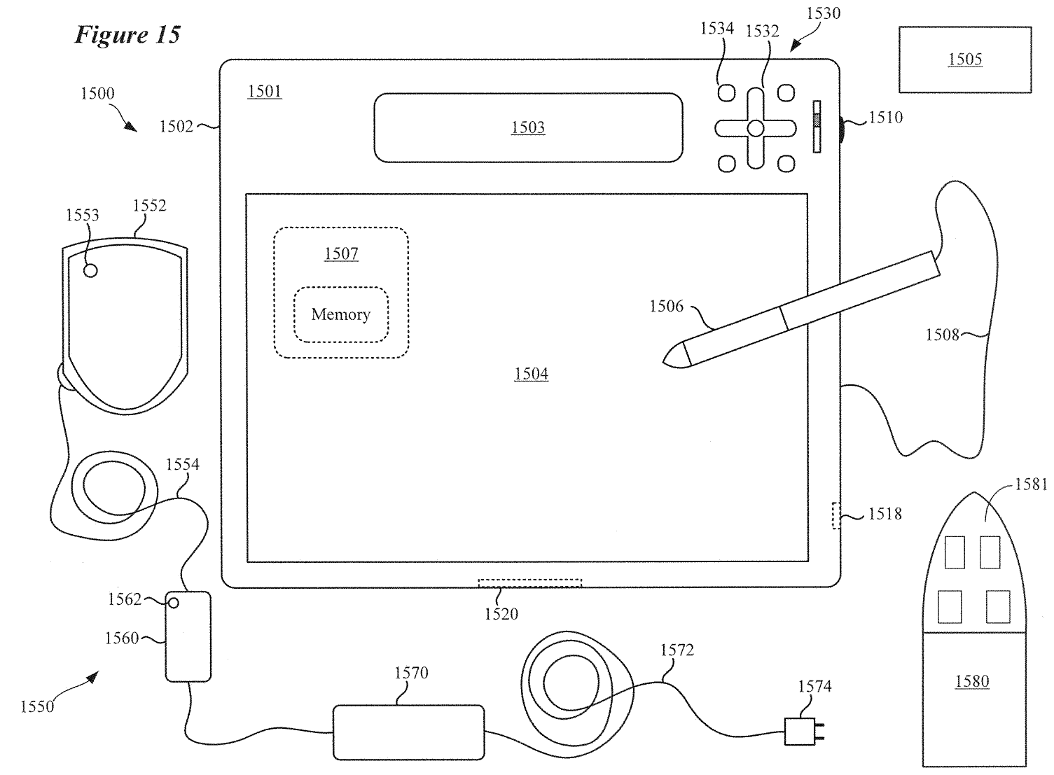

FIG. 15 shows a medical system/device that can be used to generate patient medical data which can be uploaded to a remote server in accordance with various embodiments. FIG. 15 shows a wireless programmer 1502 configured to communicate with a patient medical device 1505 via a telemetry cable 1550. According to various embodiments, the wireless programmer 1502 can be implemented as a tablet computer or other mobile computing device (e.g., a phablet, notebook or laptop). The wireless programmer 1502 is configured to implement an application (also referred to as an “app”) or a browser that facilitates clinician interaction with the telemetry cable 1550 and the patient medical device 1505. In some embodiments, the patient medical device 1505 is a neurostimulation device configured to deliver a neurostimulation therapy for treating an obstructive disordered breathing condition of the patient. In such embodiments, the neurostimulation device 1505 includes a neurostimulator and a stimulation lead that extends from the housing of the neurostimulator to the hypoglossal nerve in the patient's neck. A sensing lead extends from the housing of the neurostimulator and is implanted at an intercostal muscle location of the rib cage. The sensing lead detects intercostal muscle movement during patient respiration, signals from which are used to detect patient respiration. A pulse generator in the neurostimulator provides electrical stimulation to the hypoglossal nerve via the stimulation lead based on detected patient respiration.

The wireless programmer 1502 can be used by a clinician to interrogate the patient medical device 1505 and make adjustments to various parameters (referred to as “programming”), monitor therapy delivered by the patient medical device 1505, and monitor patient adherence to prescribed therapy. FIG. 15 also shows a wireless patient remote 1580 configured to facilitate patient adjustment of one or more therapy settings of the patient medical device 1505 directly (i.e., without the need of programmer 1502).

The telemetry cable 1550 communicates wirelessly with the patient medical device 1505 and facilitates wireless communication between the patient medical device 1505 and the wireless programmer 1502. The wireless programmer 1502 includes a display 1504 and a stylus 1506 which allows the clinician to interact with the display 1504, such as by inputting, modifying, and reviewing data. In some embodiments, the display 1504 can be configured as a touchscreen, in which case the stylus 1506 may be excluded or an optional accessory. The wireless programmer 1502 includes a number of interfaces, buttons, and controls, several of which are shown in the illustrative embodiment of FIG. 15. A power button 1510 is provided on an upper right edge of the housing 1501, and a cluster of controls 1530 is provided on an upper right portion of the front surface of the housing 1502. The control cluster 1530 includes a multi-position control 1532 that allows the clinician to interact with a processor 1507 and display 1504 of the programmer 1502 in various ways. The processor 1507 of the programmer 1502 can be programmed to implement the various processes and functions described herein. Additional buttons 1534 can be situated proximate (or apart from) the control cluster 1530. The wireless programmer 1502 includes a number of different interfaces/components including a power connector plus USB port 1518 and a network cable and USB port 1520. The interfaces and components listed above are for purposes of illustration, not of limitation.

The telemetry cable 1550 is configured to wirelessly communicate with both the wireless programmer 1502 and the patient medical device 1505. The telemetry cable 1550 effectively serves as a wireless bridge or modem between the programmer 1502 and the patient medical device 1505. In accordance with the embodiment shown in FIG. 15, the telemetry cable 1550 includes a telemetry head 1552 configured to wirelessly communicate with the patient medical device 1505 via a near-field link. The telemetry head 1552 is shown to include a status indicator 1553, such as an LED indicator. In some embodiments, the telemetry head 1552 is configured to inductively communicate with the patient medical device 1505 via a near-field link.

A cable 1554 extends from the telemetry head 1552 and is connected to a wireless transceiver 1560. The wireless transceiver 1560 may be configured for short-range radio frequency (RF) communication. For example, the wireless transceiver 1560 may be configured to implement a short-range RF communication link, such as by implementing a Bluetooth® or ZigBee® communications protocol. In some embodiments, the wireless transceiver 1560 can be configured to wirelessly communicate with existing network infrastructure via an appropriate RF communication protocol, such as Wi-Fi®. The wireless transceiver 1560 is shown to include a status indicator 1562. Power is supplied to the telemetry cable 1550 by way of a power supply 1570, which is shown to include a power cable 1572 terminated by a standard AC wall plug 1574. The power supply 1570 provides power for both the wireless transceiver 1560 and the telemetry head 1552.

The patient remote 1580 (patient remote) is shown to include buttons to allow the patient to modify therapy parameters, and status indicators for implantable device status (e.g., remote and implantable device communication status and implantable device battery status) and remote status (remote battery status). The patient remote 1580 is utilized by a patient during home use of the therapy and to make necessary adjustments of therapy parameters (e.g., stimulation amplitude) if needed or desired. The patient remote 1580 shown in FIG. 15 has a control panel 1581 which includes a number of user actuatable control buttons. The control buttons provided on the control panel 1581 allow the patient to turn therapy on and off, pause therapy, and allow the patient to adjust one or more parameters that affect the operation of the implantable medical device that is surgically implanted in the patient. For example, the control panel 1581 can include a therapy ON button and a therapy OFF button, which can be actuated to respectively turn on and off stimulation therapy by the patient. An increase control provided on the control panel 1581 allows the patient to increase stimulation strength within a range selected by the clinician. A decrease control provided on the control panel 1581 allows the patient to decrease the stimulation strength within a range pre-selected by the clinician.

The wireless programmer 1502, patient remote 1580, and patient medical device 1505 (e.g., a neurostimulator) can be implemented in accordance with commonly-owned U.S. Pat. No. 9,839,786 (Rondoni et al.) and U.S. Pat. Pub. No. 2016/0193468 (Rondoni et al.), each of which is incorporated herein by reference.

The term patient medical device as used herein is intended to represent a broad range of external and implantable devices. A patient medical device can be a device subject to medical regulatory body approval or a consumer device, such as a wearable, which is not subject to medical regulatory body approval. A patient medical device can be a device configured to communicate with an implantable therapy or a diagnostic device (e.g., a patient remote or programmer). A patient medical device can be a device in a patient's home, bedroom, at a medical clinic, a clinician device, a wearable device, a portable device, a stationary device or an implantable device.

In this disclosure, reference is made to various processors (e.g., first, second, third, fourth, separate, server, clinician processors). A processor as disclosed herein may be a single processor or multiple processors. A processor can be implemented as one or more of a multi-core processor, a digital signal processor (DSP), a microprocessor, a programmable controller, a general-purpose computer, a special-purpose computer, a hardware controller, a software controller, a combined hardware and software device, such as a programmable logic controller, a programmable logic device (e.g., FPGA, ASIC), a personal computer (PC), a main frame computer, a laptop, a notebook, a tablet, phablet or a personal digital assistant, such as a smartphone. A processor can include or be coupled to memory, such as RAM, SRAM, ROM, flash, SSD (solid-state drive), or a hard drive (HDD). A processor can be a component of a computing or communication device that cooperates with a communication interface (e.g., wired or wireless interface), a display (e.g., a touch screen), memory (RAM, ROM, flash, SSD, hard drive), and a user interface (e.g., keyboard, mouse, voice control interface, touch screen). A processor can include or be coupled to one or more communication interfaces, such as a wired and/or wireless interface (e.g., USB, FireWire®, Lightning®, GSM, CDMA, GPRS, HDSPA, Bluetooth®, ZigBee®, IEEE 802.11, ISO/IEEE 11073 compliant interface). A processor of the present disclosure can be programmed to execute program instructions or code (e.g., software, firmware) to cause the processor to perform the processes disclosed herein, such as those described with reference to the Figures (e.g., interrogating, producing, acquiring, uploading, transmitting, aggregating, accessing, selecting, displaying, determining, authenticating, granting, sharing, encrypting).

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

What is claimed is:

1. A method, comprising:
interrogating a therapy device associated with a patient to obtain device data using a first processor, and transmitting the device data from the first processor to a remote server;
producing patient-generated data using a second processor, and transmitting the patient-generated data from the second processor to the remote server;
producing clinician-generated data using a third processor, and transmitting the clinician-generated data from the third processor to the remote server;
aggregating, by the remote server, the device data and one or both of the patient-generated data and the clinician-generated data to produce aggregated patient data;
accessing the aggregated patient data at the remote server by at least one of the first, second, and third processors or a separate processor; and
displaying the aggregated patient data on a display coupled to at least one of the first, second, and third processors or the separate processor.

2. The method of claim 1, wherein aggregating comprises aggregating, by the remote server, the device data, the patient-generated data, and the clinician-generated data to produce the aggregated patient data.

3. The method of claim 1, wherein the device data comprises therapy settings data, therapy utilization data, and therapy device diagnostic data.

4. The method of claim 3, wherein the therapy settings data comprises programmable parameter values stored in the therapy device that are unique to the patient.

5. The method of claim 3, wherein the therapy settings data comprises clinician programmable parameter values and patient programmable parameter values.

6. The method of claim 3, wherein the therapy utilization data comprises a start delay and data indicating a date and time when therapy was delivered, therapy was paused, and therapy was turned off in response to patient inputs to the therapy device.

7. The method of claim 3, wherein the device data comprises device diagnostic data.

8. The method of claim 3, wherein the device data comprises programmable stimulation settings and programmable sensing settings.

9. The method of claim 1, comprising:
acquiring physiologic data from one or more physiologic sensors and transmitting the physiologic data to the remote server; and
aggregating, by the remote server, the device data, the patient-generated data, the clinician-generated data, and the physiologic data to produce the aggregated data.

10. The method of claim 1, comprising:
selecting, by the remote server, a subset of the aggregated patient data for a specific point in time; and
displaying the subset of aggregated patient data on the display.

11. The method of claim 1, comprising:
selecting, by the remote server, a subset of the aggregated patient data for a specified span of time; and
displaying the subset of aggregated patient data on the display.

12. The method of claim 11, comprising:
determining, by the remote server, the specified span of time using an interval between patient visits to a clinician or a medical practice.

13. The method of claim 1, comprising:
selecting, by the remote server, a subset of the aggregated patient data for a specific point in time or a specified span of time; and
displaying the subset of aggregated patient data on the display;
wherein the subset of aggregated patient data comprises therapy utilization data, therapy device settings, changes to clinician programmable parameter values stored in the therapy device, and changes to patient programmable parameter values stored in the therapy device.

14. The method of clam 1, comprising transforming the aggregated patient data from a first format or structure to a second format or structure appropriate for the display.

15. A system, comprising:
a remote server comprising:
a communication interface configured to communicate with a first processor configured to produce therapy device data, a second processor configured to produce patient-generated data, and a third processor configured to produce clinician-generated data;
memory configured to store the therapy device data received from the first processor, the patient-generated data received from the second processor, and the clinician-generated data received from the third processor;
a data transformation module; and
a server processor coupled to the communication interface, the memory, and the data transformation module, the server processor configured to:
aggregate the therapy device data and one or both of the patient-generated data and the clinician-generated data to produce aggregated patient data;
grant access to the aggregated patient data by at least one of the first, second, and third processors or a separate processor; and
cooperate with the data transformation module to format the aggregated patient data for presentation on a display coupled to at least one of the first, second, and third processors or the separate processor.

16. The system of claim 15, wherein the server processor is configured to aggregate the therapy device data, the patient-generated data, and the clinician-generated data to produce the aggregated patient data.

17. The system of claim 15, wherein:
the therapy device data comprises therapy settings data, therapy utilization data, and therapy device diagnostic data; and
the therapy settings data comprises clinician programmable parameter values and patient programmable parameter values.

18. The system of claim 17, wherein the therapy utilization data comprises a start delay and data indicating a date and time when therapy was delivered, therapy was paused, and therapy was turned off in response to patient inputs to a therapy device.

19. The system of claim 15, wherein:
the communication interface is configured to communicate with a sensor processor configured to produce physiologic data generated by one or more physiologic sensors;
the memory is configured to store the physiologic data received from the sensor processor; and
the server processor is configured to aggregate the physiologic data, therapy device data, and one or both of the patient-generated data and the clinician-generated data to produce the aggregated patient data.

20. The system of claim 15, wherein the server processor is configured to:
select a subset of the aggregated patient data for a specific point in time or a specified span of time; and
cooperate with the data transformation module to format the subset of aggregated patient data for presentation on the display.

21. The system of claim 20, wherein the subset of aggregated patient data comprises therapy utilization data, therapy device settings, changes to clinician programmable parameter values stored in the therapy device, and changes to patient programmable parameter values stored in a therapy device.

22. The system of clam 15, wherein the data transformation module is configured to transform the aggregated patient data from a first format or structure to a second format or structure appropriate for the display.
